(19) United States
(12) Patent Application Publication
(10) Pub. No.: US 2009/0238810 A1
(43) Pub. Date: Sep. 24, 2009

(54) OPHTHALMIC COMPOSITION

(75) Inventor: Aungkyaw NYUNT, Kasugai-Shi (JP)

Correspondence Address: BURR & BROWN, PO BOX 7068, SYRACUSE, NY, 13261-7068, US

(73) Assignee: Menicon Co., Ltd., Nagoya-Shi (JP)

(21) Appl. No.: 12/471,692

(22) Filed: May 26, 2009

Related U.S. Application Data
(63) Continuation of application No. PCT/JP2006/324413, filed on Nov. 30, 2006.

(51) Int. Cl. A61K 31/197; C07K 14/775

(52) U.S. Cl. 424/94.1; 530/359

(57) ABSTRACT

An ophthalmic composition is provided which is capable of enhancing a barrier function of a lipid layer that constitutes a tear film, for thereby restricting and curing corneal disorders such as dry eye. It is also an object of the invention to provide the ophthalmic composition with anti-inflammatory and antimicrobial effects for thereby advantageously preventing and easing corneal/conjunctival disorders such as keratoconjunctivitis. In the present invention, apolipoprotein A-1 is included, as an active ingredient, in the ophthalmic composition.

This application is a continuation of the International Application No. PCT/JP2006/324413, filed Nov. 30, 2006, the entireties of which are incorporated herein by reference.

### BACKGROUND OF THE INVENTION

1. Field of the Invention

The present invention relates to an ophthalmic composition. In particular, the present invention relates to an ophthalmic composition which is effective in easing (treating) corneal/conjunctival disorders such as dry eye and keratoconjunctivitis.

2. Discussion of Related Art

Recently, an increased number of patients complain of dry eye symptoms, and a considerable number of patients suffer from dry eye. Described in detail, the dry eye symptoms affect 10% of the population aged between 30 and 60, and affect as many as 15% of the population aged more than 65. The numbers of people affected by the dry eye symptoms are estimated as 10-14 million in the United States and as approximately 8 million in Japan. Also, an increased number of contact lens wearers suffer from dry eye, and dry eye is the major cause of discontinuation of wearing the lens.

Dry eye is a disorder of a tear film due to tear deficiency or excessive tear evaporation which causes damage to an interpalpebral ocular surface (i.e. an exposed eye surface) and is associated with symptoms of ocular discomfort (Lemp M A Report of the National Eye Institute/Industry workshop on Clinical Trials in Dry Eyes. CLAO J, 21: p 221-232, 1995). Dry eye results from decreased secretion and increased evaporation of tear (lacrimal fluid). There are reported that hyperosmolarity of the tear film in keratoconjunctivitis sicca (KCS) may play a serious role in inducing diseases seen in a cornea and conjunctiva (Arch Opthalmology, 96(4): p 677-681, 1978), and elevated tear film osmolarity can directly cause and promote diseases on an ocular surface with dry eye (Acta Opthalmologica, 61: p 108-116, 1983, Opthalmology, 92: p 646-650, 1985, Am. J. Opthalmology, 102: p 505-507, 1986, Invest Opthalmol. Vis. Sci. 29: p 374-378, 1988). Mechanisms for increasing the osmolarity, which may be a cause of developing dry eye diseases, are shown in FIG. 1. As it is apparent from FIG. 1, increased tear osmolarity is generally caused by (1) decreased secretion and (2) increased evaporation. Moreover, the former condition, (1) decreased secretion, is caused by lacrimal gland disease, or by decreased corneal sensation, while the latter condition, (2) increased evaporation, is caused by increased palpebral fissure or by meibomian gland dysfunction (see W. B. Saunders Company, Philadelphia, In Albert D M, Jakobiec F A (eds): Principles and Practice of Opthalmology, p 257-276, 1994). In addition, especially in those patients with dry eye symptoms, hyperosmolarity of the tear film has been postulated to be a key factor in pathogenesis and diagnosis of KCS (Adv. Exp. Med. Biol., 350: p 495-503, 1994).

Moreover, the dry eye symptoms are frequently existed in patients affected with allergic conjunctivitis, and this may be an evidence that there is a possible correlation between dry eye and allergic conjunctivitis and other ocular inflammation (Dry eye clinic, Editor-K Tsubota, p 26-27, Published in 2000, Igakushoin, Japanese edition). It has been reported that there are a number of different inflammatory cytokines, including interleukin (IL)-1, IL-6, IL-8, and TNF-α elevated in conjunctival epithelium of patients with Sjögren's syndrome (SS), keratoconjunctivitis sicca (KCS), or a severe type of dry eye syndrome, compared with normal subjects (Invest Opthalmol Vis. Sci., Vol. 42(10): p 2283-2292, 2001). For this reason, once the barrier function of the ocular surface is deteriorated due to reduction of tear secretion, there is an increased risk of ocular inflammation which may in turn be chronic by the effects of antigen and inflammatory cytokine.

Meanwhile, the tear film which covers the conjunctival epithelium is formed by three layers, i.e., a mucin layer (mucous layer), a lacrimal or an aqueous layer, and a lipid layer in order of mention starting from the side of the conjunctival epithelium. There is revealed that characteristics, thickness and uniformity of the lipid layer, which is the outermost layer of these three layers, have an important role in the tear film stability. The lipid layer is composed mainly of lipid secreted from meibomian glands located in an eyelid, and has a barrier function to prevent the tear evaporation from the conjunctival epithelium and an invasion of allergen etc. If the function of meibomian glands is deteriorated and an amount of secretion of the lipid is decreased, the lipid layer is diminished and the tear (lacrimal layer) is evaporated. Accordingly, the tear osmolarity is increased, whereby the ophthalmic disorders such as dry eye is caused.

It has been reported that the functions of meibomian glands can be influenced by a level of storage content of free cholesterol (FC) and a cholesterol ester (CE) in the glands themselves (via an activity of cholesterol acyltransferase: ACAT-1, which is an enzyme that esterifies cholesterol in cells). A reduction of accumulation and storage content of CE may cause atrophic changes in meibomian glands (J. Biol. Chem., 275(28): p 21324-21330, 2000). Accordingly, there is regarded that cholesterol ester level in the cells controls atrophy or shrinkage of meibomian glands. Moreover, there is apolipoprotein C-1 (ApoC1) which is a kind of apolipoproteins that are proteins related to metabolism of lipid, and meibomian glands have been found to be severely atrophic in ApoC1 transgenic mice overexpressing human apolipoprotein C-1 (J. Clin. Invest., 101(1): p 145-152, 1998). The atrophy of meibomian glands reduces the secretion of lipid, so that it is desirable to stimulate esterification of cholesterol to improve the function of meibomian glands.

After an extensive research, inventor of the present invention hypothesized that there would be interrelationships in between the apolipoprotein and pathogenesis of dry eye. The apolipoproteins are classified into various types such as apolipoprotein A-1 (ApoA1), A2, A5, B, D, and E, in addition to the above-mentioned ApoC1, according to structures and functions thereof. Among these types of apolipoproteins, the inventor of the present invention especially paid attention to ApoA1 which is a main component of high-density lipoprotein (HDL), based on results of experiments utilizing RT-PCR analysis, which will be described later.

There is known that ApoA1 activates lecithin cholesterol acyltransferase (LCAT) which is responsible for the esterification of cholesterol, and the activation of LCAT enhances cholesterol transport of HDL, so that ApoA1 is regarded as being effective for prevention of cardiac diseases such as arteriosclerosis and cardiac infarction. In addition, hyperosmotic stress, which is regarded as a cause of dry eye, promotes ocular surface inflammation by stimulating expression and production of IL-1β and TNF-α. Many researchers have reported that ApoA1 has anti-inflammatory effects by enhancing the expression of anti-inflammatory cytokine, IL-10, and via the inhibitory action on TNF-α, IL-1β, and IL-6 (Blood, 15, 97(8): p 2381-2389, 2001, Circulation, 106: p 1127-1132, 2002, Circulation, 103(1): p 108-112, 2001, Bio. Chem. Biophys. Acta, 13; 1623(2-3): p 120-128, 2003, Lipids, 37(9): p 925-928, 2002, Ann NY Acad Sci, 966: p 464-473, 2002, Infection and Immunity, Vol. 61, No. 12, p 5140-5146, 1993). Additionally, ApoA1 is a major contributor for the anti-endotoxin function of HDL which binds lipopolysaccharide (LPS or endotoxin) and both HDL and ApoA1 neutralizes LPS-toxicity and reduces the induction of cytokine production (Acta Biochim. Biophys. Sin. (Shanghai), 36(6): p 419-424, 2004, Eur. J. Biochem., 269: p 5972-5981, 2002, Proc. Natl. Acad. Sci. USA, 90: p 12040-12044, 1993, Circ. Res., 97 (3): p 236-243, 2005). This action may play a protective role in pathogenesis and manifestation of Gram-negative infections. Moreover, ApoA1 has inhibitory action on Staphylococcus Epidermidis and herpes simplex virus (HSV)-induced cell fusion (Mol Cell Biochem., 119(1-2): p 171-178, 1993, Virology, 176(1): p 48-57, 1990, J. Cell Biochem., 45(2): p 224-237, 1991).

JP-A-9-227401 proposes a therapeutic agent for treating a corneal disorder including, as an active ingredient, an apolipoprotein J (ApoJ) which is classified into a group different from that of ApoA1. JP-A-9-227401 explains that ApoJ is related to a secretion of mucin, which forms a mucin layer of the three layers constituting the tear film. However, JP-A-9-227401 does not disclose a barrier function of the lipid layer.

### SUMMARY OF THE INVENTION

The present invention was developed in the light of the above-described situations. It is therefore an object of the invention to provide an ophthalmic composition which is capable of enhancing a barrier function of a lipid layer which constitutes a tear film, for thereby preventing and easing a corneal disorder such as dry eye. It is also an object of the invention to provide the ophthalmic composition with anti-inflammatory and antimicrobial effects, for thereby advantageously preventing and easing corneal/conjunctival disorders such as keratoconjunctivitis.

In an effort to attain the above objects, the inventor of the present invention investigated an effect of a hyperosmolarity, which is caused on an ocular surface with dry eye, to an apolipoprotein. Described in detail, an apolipoprotein mRNA gene expression under the hyperosmolarity was investigated by using RT-PCR. As a result, an expression level of the expressed apolipoprotein A-1 was reduced under the hyperosmolarity. The inventor of the present invention made a further study, and found that the corneal disorder caused by dry eye was prevented and eased, by an administration of a solution comprising apolipoprotein A-1 to the eye.

The present invention was made based on the findings described above. The present invention provides an ophthalmic composition comprising an apolipoprotein A-1 as an active ingredient.

In preferred forms of the ophthalmic composition according to the present invention, there is adopted an arrangement, in which the ophthalmic composition further comprises at least one vitamin B5 compound, and there is preferably adopted D-pantethine as the at least one vitamin B5 compound.

Further, in additional preferred forms of the present invention, the ophthalmic composition is used for treating a corneal disorder. Examples of the corneal disorder include dry eye, and dry eye can be effectively treated by the ophthalmic composition according to the present invention.

In addition, in additional preferred forms of the present invention, the ophthalmic composition is used as eye drops or as a solution for a contact lens.

In additional preferred forms of the ophthalmic composition according to the present invention, the apolipoprotein A-1 is included in the composition in a concentration of 0.001 to 1 w/v %, while the at least one vitamin B5 compound is included in the composition in a concentration of 0.005 to 2 w/v %.

As described above, the ophthalmic composition of the present invention includes the apolipoprotein A-1, so that the barrier function of the lipid layer is enhanced, and the corneal disorder such as dry eye is effectively prevented and eased.

Also, anti-inflammatory and antimicrobial effects of the apolipoprotein A-1 are also exhibited on the ocular surface, so that corneal/conjunctival disorders such as keratoconjunctivitis can be advantageously prevented and eased.

Moreover, if the apolipoprotein A-1 and the vitamin B5 compound such as D-pantethine are used together, according to the preferred form of the present invention, corneal/conjunctival disorders such as dry eye and keratoconjunctivitis are effectively prevented and eased, owing to a synergistic effect of the two components.

### BRIEF DESCRIPTION OF THE DRAWING

The above and other objects, features, advantages and technical and industrial significance of the present invention will be better understood by reading the following detailed description of presently preferred embodiments of the invention, when considered in connection with the accompanying drawings, in which:

FIG. 1 is a drawing which is obtained by modifying a drawing from W.B Saunders Company, Philadelphia, In Albert D M, Jakobiec F A (eds): Principles and Practice of Opthalmology, (p 257-276, 1994), which shows mechanisms of increased tear osmolarity which leads to a development of the dry eye diseases;

FIG. 2 is a photograph showing gel electrophoresis of apolipoprotein gene expression;

FIG. 3 is a graph relatively showing ApoA1 mRNA expressions in HCET cells stimulated by hyperosmotic stress;

FIG. 4 is a graph relatively showing ApoA5, ApoE and ApoC1 mRNA expressions in HCET cells stimulated by hyperosmotic stress;

FIG. 5 is a graph showing results of a phenol red thread test for mice which were subjected to a subcutaneous injection of scopolamine hydrobromide and to a blower drying treatment (DE mice);

FIG. 6 is a graph showing results of a phenol red thread test for DE mice which were treated with ApoA1;

FIG. 7 is a graph showing results of a phenol red thread test for DE mice which were treated with ApoA1 and D-pantethine;

FIG. 8 is a graph showing corneal staining scores of six DE mice (mouse 1 to 6) which were treated with eye drops including ApoA1 or with PBS including none of active ingredients;

FIG. 9 is a graph showing corneal staining scores of six DE mice (mouse 1 to 6) which were treated with eye drops including ApoA1 and D-pantethine (Dp) or with PBS including neither of these active ingredients;

FIG. 10 is a graph showing corneal staining scores of DE mice treated with PBS including none of active ingredients or with eye drops including ApoA1, and indicating effects of various concentrations of ApoA1;

FIG. 11 is a graph showing corneal staining scores of DE mice treated with PBS including none of active ingredients or with eye drops including ApoA1 and D-pantethine, and indicating effects of various concentration of D-pantethine;

FIG. 12 is set of photographs of corneal epithelium stained with hematoxylin and eosin, wherein (a) is a photograph of corneal epithelium of a normal mouse, (b) is a photograph of corneal epithelium of a DE mouse, (c) is a photograph of corneal epithelium of a DE mouse treated with ApoA1, and (d) is a photograph of corneal epithelium of a DE mouse treated with PBS;

FIG. 13 is a graph showing respective average corneal epithelial thicknesses of respective groups of normal mice, DE mice, DE mice treated with PBS, and of DE mice treated with ApoA1;

FIG. 14 is a graph showing respective average corneal epithelial thicknesses of groups of normal mice, DE mice, DE mice treated with PBS, and of DE mice treated with ApoA1 and D-pantethin;

FIG. 15 is a graph showing respective average corneal epithelial thicknesses of groups of DE mice and of mice treated with eye drops including ApoA1, and indicating effects of various concentrations of ApoA1; and

FIG. 16 is a graph showing respective average corneal epithelial thicknesses of groups of DE mice and of mice treated with eye drops including ApoA1 and D-pantethine, and indicating effects of various concentrations of D-pantethine.

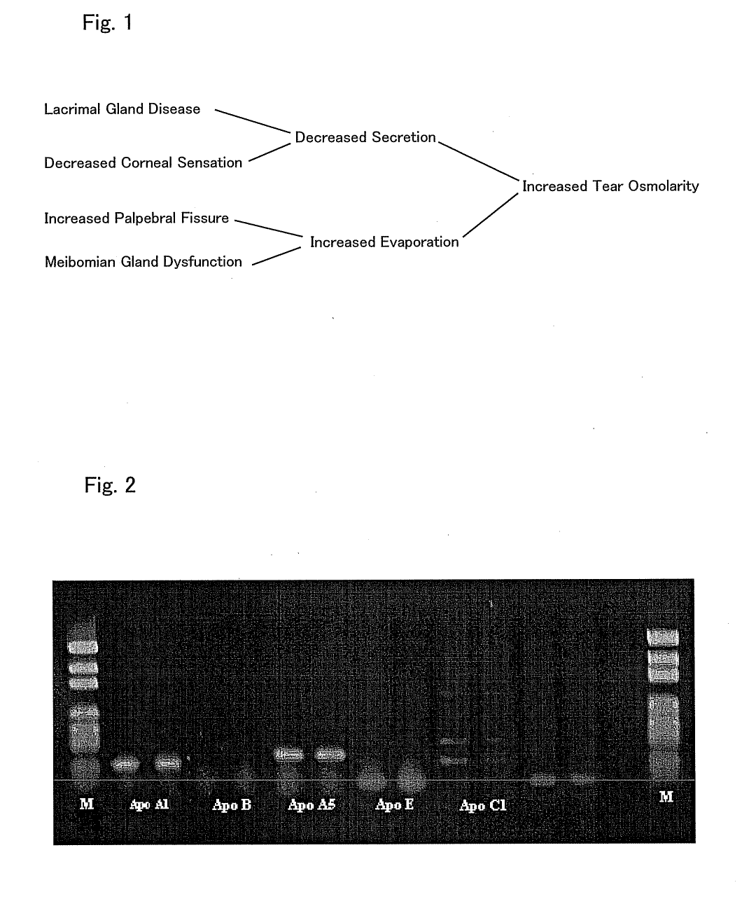

### DETAILED DESCRIPTION OF THE INVENTION

The ophthalmic composition according to the present invention is comprised of the apolipoprotein A-1 (ApoA1) as the active ingredient, and can be used as the eye drops to treat various corneal/conjunctival disorders such as dry eye, keratoconjunctivitis, allergic conjunctivitis, and microbial keratitis, and can be used as the solution for the contact lens which is brought into contact with the contact lens.

As described above, ApoA1 as the active ingredient in the present invention is a kind of apolipoproteins, which is known as possessing LCAT activation function, and as exhibiting the anti-inflammatory effect. By adopting ApoA1 as the active ingredient of the ophthalmic composition, the barrier function of the lipid layer which constitutes the tear film is advantageously improved, so that a defection of the corneal epithelium cells (a thinning of corneal epithelium) etc., are advantageously restricted, whereby symptoms of dry eye including corneal inflammation and conjunctive inflammation are effectively prevented and eased. In addition, owing to the anti-inflammatory and antimicrobial effects of the apolipoprotein A-1, there can also be effectively prevented and eased the corneal/conjunctival disorders such as the keratoconjunctivitis, allergic conjunctivitis, and microbial keratitis.

ApoA1 is commercially available in a solid form or in a form of an aqueous solution. If the ophthalmic composition according to the present invention is used by being administrated to the eye or by being caused to contact with a contact lens, ApoA1 is used in a state of a solution, especially an aqueous solution.

That is, the ophthalmic composition according to the present invention can be adopted to any dosage forms including powdery, granular, tablet, and liquid forms. However, in the use of the ophthalmic composition, there is adopted the form of the aqueous solution (liquid agent) constituted principally by an aqueous medium. Accordingly, undermentioned concentrations of respective components indicate concentrations of the components in the state of the aqueous solution at the time of use of the composition.

Concentration of ApoA1 as the active ingredient is suitably determined. However, if the concentration of ApoA1 in the composition is too low, there cannot be sufficiently obtained intended effects of preventing and easing dry eye. On the other hand, if the concentration of ApoA1 in the composition is too high, there is an anxiety that the ophthalmic composition may easily be bubbling or foaming. Accordingly, it is desirable that ApoA1 is included in the composition in a concentration of 0.001 to 1 w/v %, preferably 0.01 to 0.5 w/v %, more preferably 0.04 to 0.2 w/v %.

It is also preferable that the ophthalmic composition according to the present invention which comprises the above-mentioned ApoA1 as the active ingredient further comprises at least one vitamin B5 compound, in addition to ApoA1. This arrangement synergistically enhances effects of ApoA1 to prevent and ease dry eye.

Examples of the vitamin B5 compounds include: pantothenic acid or salts thereof, pantethine, pantethein, panthenol, pantothenyl ethyl ether, acetylpantothenyl ethyl ether, and any one of, or any combination of these compounds are used. Among these compounds, pantethine is especially preferably adopted to the composition.

Pantethine is a derivative of vitamin B5, and is a precursor to coenzyme A (CoA) which is involved in oxidative metabolism. Pantethine supports lipid metabolism by its ability to raise levels of CoA, a cofactor involved in several metabolic pathways including carbohydrate, lipid and amino acid metabolism. CoA combines with acetyl groups to form Acetyl CoA, an essential factor in energy production. In addition, pantethine increases a serum level of high density cholesterol (HDL) and decreases the level of low density cholesterol (LDL). It also has the enhancing effects on re-epithelialization including corneal epithelium. Accordingly, there is expected that an instillation of pantethine-containing artificial tears would remarkably improve disturbances of the corneal epithelium permeability.

Concentration of the above-mentioned vitamin B5 compound in the composition is suitably determined, and is not particularly limited. However, if the concentration of the vitamin B5 compound in the composition is too low, the effect by the use of the composition is not sufficiently exhibited. On the other hand, if the concentration of the vitamin B5 compound in the composition is too high, viscosity of the composition is increased, which may interrupt a blink of the eye. Therefore, it is desirable that the concentration of the vitamin B5 compound in the composition is within a range of 0.005 to 2 w/v %, preferably 0.05 to 0.5 w/v %, more preferably 0.1 to 0.2 w/v %.

In adding the vitamin B5 compound, there is not particularly limited a ratio of ApoA1 and the vitamin B5 compound. However, it is desirable that the ratio of ApoA1 and the vitamin B5 compound is 4:5 to 1:5, preferably 2:5 to 1:5. By adopting these ratios, there can be further efficiently obtained the synergistic effects of the two components.

The ophthalmic composition according to the present invention may further contain, as needed, any one of, or any combination of a conventionally adopted ratio of various known additives as used in conventional ophthalmic compositions such as eye drops and solutions for a contact lens, in addition to ApoA1 and the vitamin B5 compound. The additives to be included in the composition should be safe to the living body and ophthalmically and physiologically acceptable, and should not give adverse influences on a configuration and physical properties of the contact lens. The additives are included in the composition in amounts that fulfill the above-described requirements, and by adopting this arrangement, there can be advantageously provided various effects given by the additives to the ophthalmic composition without deteriorating the effect of the present invention.

For instance, in the ophthalmic composition of the present invention, if the osmolarity of the composition is too high or too low, there is an anxiety of causing a stimulus to the eye or causing other problem to the eye. For this reason, it is generally desirable that the osmolarity is adjusted to be in a range of about 200 to about 350 mOsm/L, by adding an isotonic agent, etc. Examples of the isotonic agent include sodium chloride, potassium chloride, sugars, sugar alcohols, and polyols and ethers or esthers thereof. Any one of, or any combination of these isotonic agents are used.

In the ophthalmic composition of the present invention, if a pH value of the composition is too high or too low, there is an anxiety of causing irritation to the eye or causing the eye damage. For this reason, it is generally desirable that the pH value of the composition is adjusted to be held in a range of about 5.3 to about 8.5, preferably around 7.0, by adding a suitable pH value adjusting agent and/or a buffer. Examples of the pH adjusting agent to be used to adjust the pH value of the composition include sodium hydroxide and potassium hydroxide. Meanwhile, examples of the buffer to effectively adjust the pH value to the above-mentioned range which assures safety to the eye include: acids such as phosphoric acid, boric acid, carboxylic acid, oxycarboxylic acid, and salts thereof (such as sodium salts); a Good-Buffer, tris (hydroxymethyl) aminomethane (TRIS), bis(2-hydroxyethyl) iminotris (hydroxymethyl) methane (Bis-Tris) and sodium hydrogen carbonate. These buffers are used, because they are safe to the eyes and are able to reduce influences to the contact lenses. The buffer for the ophthalmic composition of the present invention is suitably selected from among the above-mentioned known buffers.

Moreover, for permitting the ophthalmic composition of the present invention to advantageously exhibit a disinfecting efficacy for the eye or the contact lens and to further exhibit a preservative efficacy of the ophthalmic composition, a preservative or a disinfectant which has a preserving or a disinfecting effect is added to the composition. It is generally desirable that the preservative or the disinfectant has an excellent compatibility with the eye or the contact lens, as well as the preserving or disinfecting effect, and is less likely to cause troubles such as an allergy. Any one of, or any combination of conventionally known preservative or disinfectants may be suitably selected and used.

Examples of the preservative include sorbic acid, potassium sorbate, benzoic acid or salts thereof, ethyl parahydroxybenzoate, butyl parahydroxybenzoate, propyl parahydroxybenzoate, methyl parahydroxybenzoate, chlorobutanol, and perbotates such as perboric acid or sodium perborate. Meanwhile, as the disinfectant, a biguanide compound such as polyhexamethylene biguanide (PHMB) or a polymeric quaternary ammonium compound such as polyquaterium can be used. In using the ophthalmic composition of the present invention in a form of eye drops without adding the above-mentioned preservative or disinfectant, the ophthalmic composition of the present invention may be used in a single dose type eye drop, in which the eye drops are used off in one administration, or may be used in a multi dose type eye drop application, with which a dispenser with a filter is used.

Generally, there is a possibility that deposits from the lacrimal fluid such as calcium may adhere to the contact lenses, especially, to the soft contact lenses. For this reason, a chelating agent may also be included in the present ophthalmic composition, so as to prevent accumulation or adsorption of the deposits such as calcium. Examples of the chelating agent include ethylenediamine tetraacetic acid (EDTA) and salts thereof, such as disodium salts of ethylenediamine tetraacetic acid (EDTA.2Na), and trisodium salts of ethylenediamine tetraacetic acid (EDTA.3Na).

In addition, in order to remove or to clean stain of eye lipid adhered to the cornea or the contact lens, any known anionic surfactants, amphoteric surfactants, cationic surfactants, and nonionic surfactants may be added to the ophthalmic composition of the present invention.

Examples of the surfactant include: polyglycerin fatty acid ester, polyoxyethylene alkylether, polyoxyethylne-polyoxypropylene block copolymer, polyoxyethylene-polyoxypropylene ethylenediamine, polyoxyethylene sorbitan fatty acid ester, condensation products of polyoxyethylene alkylphenyl ether and formaldehyde, polyoxyethylene hardened castor oil, polyoxyethylene alkylphenyl ether, polyoxyethyleneglycerin fatty acid ester, polyoxyethylene sorbitan fatty acid ester, polyoxyethylene castor oil, polyoxyethylene sterol, polyoxyethylene hydrogenated sterol, polyoxyethylene fatty acid ester, polyoxyethylene-polyoxypropylene alkylether, polyoxyethylene lanolin alcohol, polyoxyethylene alkylamine, polyoxyethylene alkylamide, polyoxyethylene alkyl ether phosphoric acid, and polysorbate, and any one of, or any combination of these surfactants may be used.

The present ophthalmic composition may further contain a thickener, in order to suitably adjust the viscosity of the composition, to extend the period of time for the ophthalmic composition to stay on the corneal surface in the conjunctival sac, and further to improve wettability and moisture retaining property. By the addition of the thickener, the effect of the active ingredient added to the ophthalmic composition is maintained, and the effect of reducing dryness etc. of the eye can be exhibited for an extended period of time. Examples of the thickener include: gluconic acid and salts thereof; mucopolysaccharides such as chondroitin sulfate and salts thereof; hyaluronic acid and salts thereof; various gums such as heteropolysaccharides and polysaccharides; synthetic organic high molecular compounds such as polyvinyl alcohol, poly-N-vinylpyrrolidone, polyethylene glycol, polypropylene glycol, polyacrylamide; cellulose derivatives such as cationized celluloses including polyquaterium-10, hydroxy ethyl cellulose, hydroxypropyl methylcellulose, carboxymethyl cellulose, and methyl cellulose; and starch derivatives, and these thickeners are advantageously used.

Moreover, for the purposes of providing a refreshing sensation at the time of the administration of the composition to the eye, resolving discomfort and itchiness at the time of wearing the lens, and so on, refreshing agents such as menthol, borneol, camphor, geraniol, eucalyptus oil, bergamot oil, fennel oil, peppermint oil, rose oil, or coolmint may be added to the present composition.

In addition to the above, the ophthalmic composition in accordance with the present invention may include: vitamins such as vitamin A compounds such as retinol palmitate and β-carotene, vitamin B₂, B₆, B₁₂, and vitamin E compounds such as d-α tocopherol acetate; vasoconstrictive agents such as naphazoline nitrate and tetrahydrozoline hydrochloride; amino acids such as aspartic acid and salts thereof, aminoethylsulfonic acid, arginine, alanine, lysine, and glutamic acid, according to an intended use of the composition.

The ophthalmic composition of the present invention is prepared by adding and including respective suitable amounts of the above-mentioned components to the suitable aqueous medium, according to a conventionally known method. It is needless to mention that, in addition to water itself such as a tap water and a purified water, there can also be used any solution, such as a saline solution and a buffer solution, as long as the solution includes water as its main component and the solution assures a high degree of safety to the living body, and can be opthalmologically sufficiently accepted.

In preparing the ophthalmic composition of the present invention, the ophthalmic composition can be easily obtained by simply dissolving the respective components in the aqueous medium, similar to a preparation of a conventional solution, without requiring any special method. Also, in preparing the ophthalmic composition in the powdery, granular, or tablet form, other than the liquid agent form, the ophthalmic composition may be at first prepared in the form of the liquid agent as described above, and then the ophthalmic composition in the form of the liquid agent is dehydrated by a known drying method, such as freeze dehydration or reduced-pressure drying. If the ophthalmic composition is prepared in the solid form as described above, there is a merit that the ophthalmic composition can be more easily preserved. The ophthalmic composition in the solid form is dissolved in a predetermined amount of the aqueous solution, so as to fulfill the above-mentioned preferred range of the concentration, just before the use of the ophthalmic composition. If the ophthalmic composition is prepared in the liquid form, the ophthalmic solution may be diluted just before the use of the ophthalmic composition, so as to fulfill the above-mentioned preferred range of the concentration.

The present ophthalmic composition obtained as described above is effective for the corneal disorders such as dry eye and keratoconjunctivitis, and the composition does not cause any adverse effect such as change of configuration of the contact lens, so that the present composition can be advantageously used as eye drops and as the solution for the contact lens, such as an ophthalmic solution for the contact lens and a multi-purpose solution for the contact lens.

For instance, if the ophthalmic composition of the present invention is used as eye drops, preferable amounts of the ophthalmic composition is administrated to the eye one to several times a day, similar to administrations of known ophthalmic solutions or eye drops.

If the ophthalmic composition of the present invention is administrated to the eye, esterification of cholesterol in the cells is promoted by ApoA1, whereby the functions of meibomian glands are improved. Accordingly, the barrier function of the lipid layer is effectively exhibited. Thus, the corneal disorders such as dry eye and keratoconjunctivitis are restricted and relieved. Moreover, owing to the anti-inflammatory and antimicrobial effects of ApoA1, corneal/conjunctival disorders such as keratoconjunctivitis, allergic conjunctivitis, and microbial keratitis are advantageously relieved or restricted.

The ophthalmic composition of the present invention does not have any adverse effect to a contact lens, so that there will be no problem even if the contact lens is still worn on the eye, at a time of administrating the ophthalmic composition to the eye.

If the ophthalmic composition of the present invention is used as the solution for the contact lens, other than eye drops, the contact lens, which has been removed from the eye, is cleaned or rinsed with the ophthalmic composition. Thereafter, the contact lens may be worn while the ophthalmic composition is still attached to surfaces of the contact lens. Instead, the contact lens may be preserved by being immersed in the ophthalmic composition for a predetermined period of time, and then the contact lens may be taken out of the ophthalmic composition and worn on the eye as it is. By using the ophthalmic composition as described above, the active ingredients in the ophthalmic composition is administrated to the eye via the contact lens, whereby the effects of the present invention are advantageously exhibited, i.e., symptoms such as dry eye are prevented and eased.

Examples of the above-mentioned solution for the contact lens include: single purpose solutions such as a contact lens cleaning solution, contact lens rinsing solution, and a contact lens storing solution; and a multi-purpose solution which is intended for several purposes such as cleaning, rinsing and storing.

If the ophthalmic composition is used as the solution for contact lens such as eye drops for the contact lens or the multi-purpose solution for contact lens, the type of the contact lens to be treated is not limited. For example, soft contact lenses, which are classified into non-water-content contact lenses, low-water-content contact lenses, and high-water-content contact lenses, and hard contact lenses can be the contact lenses to be treated with the ophthalmic composition. Therefore, the ophthalmic composition of the present invention is applied to any contact lens, regardless of the material, etc. of the contact lens.

### EXAMPLES

To further clarify the concept of the present invention, some examples of the invention will be described. It is to be understood that the invention is not limited to the details of the illustrated examples and the foregoing description, but may be embodied with various changes, modifications and improvements, which may occur to those skilled in the art without departing from the scope of the invention defined in the attached claims.

All the statistical data in the undermentioned EXAMPLES were calculated by using one-way ANOVA with Pair-Wise comparisons via Turkey test.

—In Vitro Experiment Using SV-40 Immortalized Cultured Cell Line (HCET)—

The effect of hyperosmotic stress on corneal epithelium cells and its effects on the apolipoprotein mRNA gene expressions were examined. HCET cells were stimulated with hyperosmotic culture medium (500 mOsm/L) for 3 hours, 6 hours, and overnight, incubated at 37° C. Cell culture medium was DMEM medium (Dulbecco's Modified Eagle's Medium) and compositions as follows:

| | | |
|---|---|---|
| DMEM/F12 | 95 | mL |
| FBS (Fetal Bovine Serum) | 5 | mL |
| 7.5% NaHCO₃ | 2.4 | mL |
| Antibiotic Antimycotic | 1 | mL |
| 5 mg/mL insulin | 0.1 | mL |

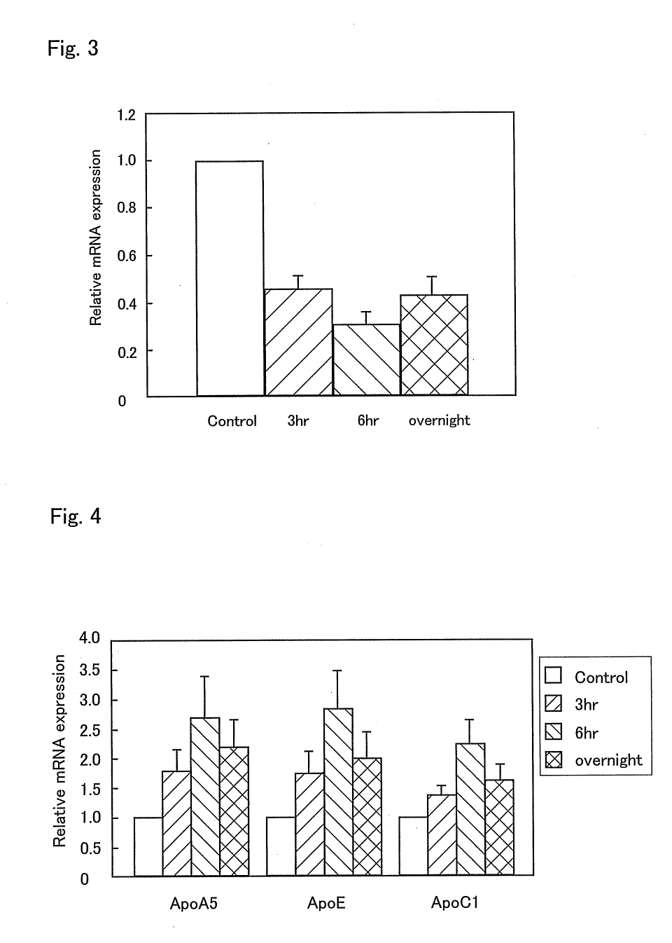

After the incubation, cells were lysed with TRizol for the RNA extraction, and then purified with phenol and ethanol precipitation. Then, mRNA gene expressions were confirmed by SAGE analysis, RT-PCR analysis, and Real-time RT-PCR analysis. Thus obtained data of SAGE analysis are shown in TABLE 1 below, results of RT-PCR analysis are shown in FIG. 2, and results of Real-time RT-PCR analysis are shown in FIGS. 3 and 4, respectively. Primers used for RT-PCR analysis and Real-time RT-PCR analysis are as follows:

<Primers used for RT-PCR analysis>
Apolipoprotein A-1:
Forward 5′-GTACGTGGATGTGCTCAAAGAC-3′
Reverse 5′-CTCCAGATCCTTGCTCATCTCT-3′
Apolipoprotein A-5:
Forward 5′-CAGATAATGGCAAGCATGGCT-3′
Reverse 5′-GTCTGGCTGAAGTAGTCCCAGAAG-3′
Apolipoprotein E:
Forward 5′-GCAGGAAGATGAAGGTTCTGTG-3′
Reverse 5′-CCTTCAACTCCTTCATGGTCTC-3′
Apolipoprotein B:
Forward 5′-AGTCTTCCTTATACCCAGACTTTGC-3′
Reverse 5′-GTACAAGTTGGTGTAGACATTCGTG-3′
Apolipoprotein C-1:
Forward 5′-CTCCAGTGCCTTGGATAAGC-3′
Reverse 5′-GGTGTGGGAAATTTCAGAGG-3′
<Primers used for Real-time RT-PCR analysis>
Apolipoprotein A-1:
Forward 5′-GCTCAAAGACAGCGGCAGAG-3′
Reverse 5′-AGGTCACGCTGTCCCAGTTG-3′
Apolipoprotein A-5:
Forward 5′-GAAAGGCTTCTGGGACTACTTCAG-3′
Reverse 5′-AGATCCATCGTGTAGGGCTTC-3′
Apolipoprotein E:
Forward 5′-GCCAATCACAGGCAGGAAGA-3′
Reverse 5′-GCTCTGTCTCCACCGCTTG-3′
Apolipoprotein C-1:
Forward 5′-CTCCAGCAAGGATTCAGAGTG-3′
Reverse 5′-CTTCAGGTCCTCATGAGTCAATC-3′

TABLE 1

| Tag Seqence | Unigene ID | Description | Hyper | Control |
|---|---|---|---|---|
| TGGCCCCAGG | Hs.110675 | apolipoprotein C-1 | 5 | 1 |
| CGACCCCACG | Hs.151465 | apolipoprotein E | 3 | 1 |

FIG. 2 shows the gel electrophoresis of the apolipoprotein gene expressions. According to these data, there can be recognized ApoA1, ApoA5, ApoE and ApoC1 were expressed in human corneal epithelial cells. On the other hand, ApoB expression was not detected. In FIG. 2, M represents a DNA marker.

In FIGS. 3 and 4, there are shown relative changes of mRNA expressions ratio to the control. In FIGS. 3 and 4, the control represents the cells which were not stimulated under a hyperosmolarity, while 3 hr, 6 hr, and overnight represent the cells which were respectively stimulated under the hyperosmolarity for 3 hours, 6 hours, and overnight. In FIG. 3, there is recognized that a degree of mRNA expressions of ApoA1 in HCET cells is reduced. On the other hand, in FIG. 4, there is recognized that degrees of mRNA expressions of ApoA5, ApoE, and ApoC1 in HCET cells are increased. Accordingly, there is confirmed that ApoA1 has a different behavior under the hyperosmolarity, compared with the other apolipoproteins. In this EXAMPLE, the Real-time RT-PCR data (fluorescent intensities) were normalized with 18S RNA. In addition, n=6 eyes and n=4 to 5 eyes were independently tested for ApoA1 and for the other apolipoproteins, respectively (mean±SD).

—In Vivo Experiment Using Experimental Mouse Model of Dry Eye—

<Mouse Model of Dry Eye>

C57BL/6J mice aged 10 to 12 weeks were used for studies. All the studies were performed in accordance with the ARVO Statement for the Use of Animals in Ophthalmic and Vision Research. Each mouse model of dry eye was created by subcutaneously injecting 0.5 mg/0.2 mL scopolamine hydrobromide (Sigma-Aldrich, St. Louis, Mo.) at the back, four times a day (q.i.d.) at 9 A.M., 12 P.M., 3 P.M., and at 6 P.M. There were also used untreated age-matched mice as normal control subjects (normal mice) (see Investigative Opthalmology and Visual Science. 45: p 4293-4301, 2004).

<Animals>

10-week-old male C57BL/6J Jms Slc mice were purchased. They were quarantined and acclimatized before experiments for one week period under standard conditions: room temperature 23±2° C., relative humidity 60%±10%.

<Procedure of Desiccation Treatment>

The mice were subcutaneously injected with 0.5 mg/0.2 ml of scopolamine hydrobromide four times a day for 5 days. Desiccating environmental stress was created by placing a mouse cage in a blower hood with continuous air flow blower (room temperature of 23±20 and relative humidity of 60%±10%). Tear production of each mouse was evaluated before the treatment, and also evaluated on third and fifth days of the treatment.

<Topical Eye Drop Application>

During the desiccation treatment, one of the eyes of each mouse was treated with eye drops including ApoA1 (Sigma-Aldrich, Japan) or with eye drops including ApoA1 and D-pantethine (Sigma-Aldrich, Japan), while PBS was administrated to the other eye of each mouse as a control. 5 μl of the respective eye drops was administered four times a day during the desiccation treatment for five days. All the ophthalmic compositions were formulated in PBS, and the osmolarity and the pH value of the compositions were adjusted to 290 to 300 mOsm/L and pH 7.4, respectively. There were prepared PBS which had a standard osmolarity (300 mOsm/L) by using 0.01M of PB and saline (125 mM) to make up PBS with normal osmolarity (300 mOsm/L). To prepare one liter of 0.01 M PBS, there are needed the followings:

| | | |
|---|---|---|
| NaCl (M.W. 58.44) | 7.3 | g (250 mOsm/L) |
| Na₂HPO₄•12H₂O (M.W. 358.14) | 3.5814 | g (30 mOsm/L) |
| NaH₂PO₄•2H₂O(M.W. 156) | 1.56 | g (20 mOsm/L) |

(1) Measurement of Aqueous Tear Volume Production

There were conducted phenol red thread tests in order to confirm whether the eyes of the mice have symptoms of dry eye, by the above-mentioned subcutaneous injection of scopolamine hydrobromide and the desiccation treatment by using the blower (hereinafter these treatments will be jointly abbreviated as “DE treatment”). Described in detail, tear production was measured with standardized phenol red-impregnated cotton threads (Zone-Quick; Menicon Co. Ltd, Japan). The threads were applied to the ocular surface in the lateral canthus for 60 seconds in the unanesthetized mouse. After 60 seconds, the length of the color change on the thread, indicating the length of the thread wetted by the tears, was measured in millimeters. Wetting length of the thread was measured to an accuracy of one millimeter.

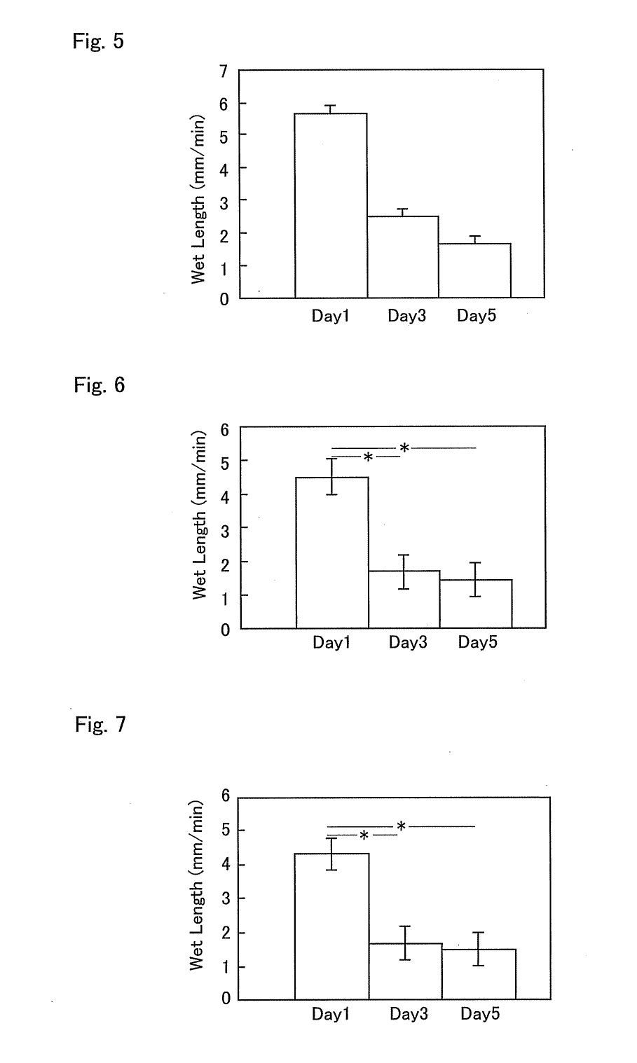

In FIG. 5, there are shown results of the phenol red thread tests of mice subjected to the DE treatment (hereinafter referred to as “DE mice”) (mean±SD, n=6 eyes), and in FIG. 6, there are shown results of the phenol red thread tests of DE mice treated with eye drops including 0.04 w/v % of ApoA1 (mean±SD, n=12 eyes, p value <0.01). Moreover, in FIG. 7, there are shown results of the phenol red thread tests of DE mice treated with eye drops including 0.04 w/v % of ApoA1, and 0.1 w/v % of D-pantethine (mean±SD, n=12 eyes, p value <0.01). In FIGS. 5 to 7, Day 1 represents a result of the test conducted before the eyes were subjected to the subcutaneous injection of scopolamine hydrobromide and the desiccation treatment (DE treatment), and Day 3 and Day 5 represent results of the tests conducted third and fifth days of the treatment, respectively.

As it is apparent from FIGS. 5 to 7, the tear secretion is remarkably reduced on Day 5, compared with that of Day 1, so that there was confirmed that the eyes of the mice had symptoms of dry eye because of the subcutaneous injection of scopolamine hydrobromide and the blower drying treatment (DE treatment).

(2) Corneal Fluorescein Staining

Corneal fluorescein staining was evaluated 10 minutes after topical application of 1 μL of 1% sodium fluorescein (Sigma-Aldrich, Japan) to mouse eyes, which were treated as described above, and examined under a light microscope (Leica Microsystems Inc., Deerfield, USA) and photographed with a digital camera (CAMEDIA, Olympus Optical Co. Ltd., Japan) fitted to the microscope.

Results of the corneal fluorescein staining were classified into grades as shown in TABLES 2 and 3 below, by using a grading system based on areas of corneal staining (Nakamura S., Tsubota K et al., Investigative Opthalmology and Visual Science. 46: p 2379-2387, 2005). In TABLES 2 and 3 below, X represents a ratio of an area which was subjected to a punctuate staining to a total area of a cornea.

TABLE 2

| Grade | 0 | 0.5 | 1 | 1.5 | 2 |
|---|---|---|---|---|---|
| Ratio of stained area | X = 0 | $0 < X ≦ \frac{1}{16}$ | $\frac{1}{16} < X ≦ \frac{1}{8}$ | $\frac{1}{8} < X ≦ \frac{3}{16}$ | $\frac{3}{16} < X ≦ \frac{1}{4}$ |

TABLE 3

| Grade | 2.5 | 3 | 3.5 | 4 |
|---|---|---|---|---|
| Ratio of stained area | $\frac{1}{4} < X ≦ \frac{3}{8} to \frac{7}{16}$ | $\frac{3}{8} to \frac{7}{16} < X ≦ \frac{1}{2}$ | $\frac{1}{2} < X ≦ \frac{5}{8} to \frac{3}{4}$ | $\frac{5}{8} to \frac{3}{4} < X ≦ 1$ |

(2-1) Effect of Eye Drops Including ApoA1

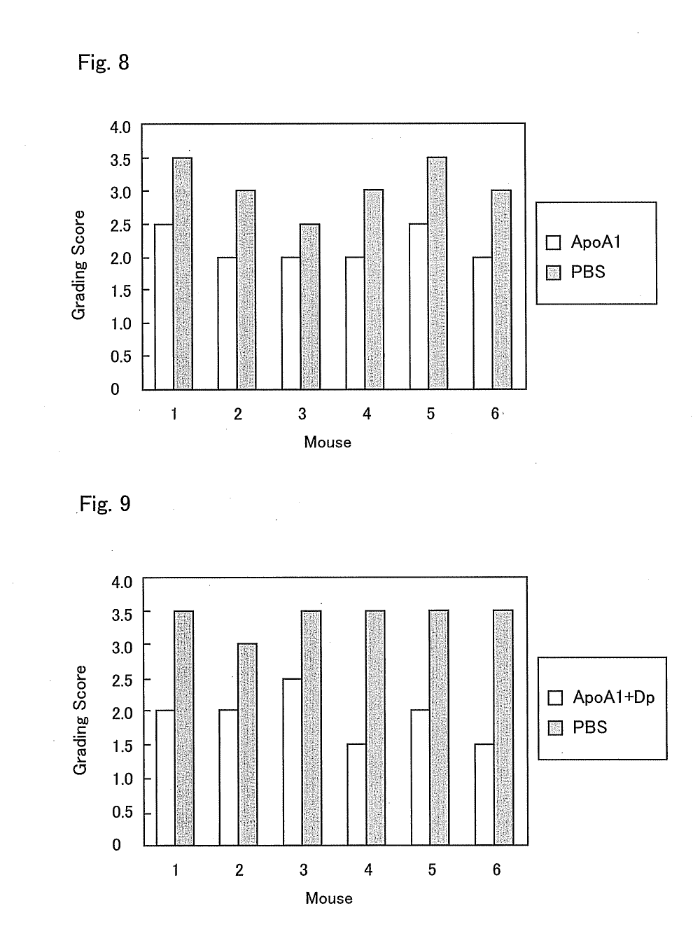

In FIG. 8, there are shown corneal staining scores of six DE mice (mouse 1 to 6) which were treated with eye drops including 0.04 w/v % of ApoA1 or with PBS (without ApoA1). According to FIG. 8, an average score of the ApoA1 group is about 25% less than that of the PBS group.

Quantitative measurement of fluorescein stained area was also performed by using an image analysis software (NIH ImageJ, version 1.36; available at http://rsb.info.nih.gov/ij/download.html). An average score of the ApoA1 group is 20% less than that of the PBS group, although the data are not shown.

According to these grading scores and the results of the quantitative measurement, ApoA1 seems to have protective effects on the corneal epithelium erosion due to desiccation.

(2-2) Effect of Eye Drops Including ApoA1 and D-pantethine

Also, in FIG. 9, there are shown corneal staining scores of six DE mice (mouse 1 to 6) which were treated with eye drops including 0.04 w/v % of ApoA1 and 0.05 w/v % of D-pantethine (Dp) or with PBS including neither of these active ingredients. Conditions of the eyes treated with ApoA1 in combination with D-pantethine are remarkably improved, compared with the eyes treated with PBS.

(2-3) Effect of Various Concentrations of ApoA1

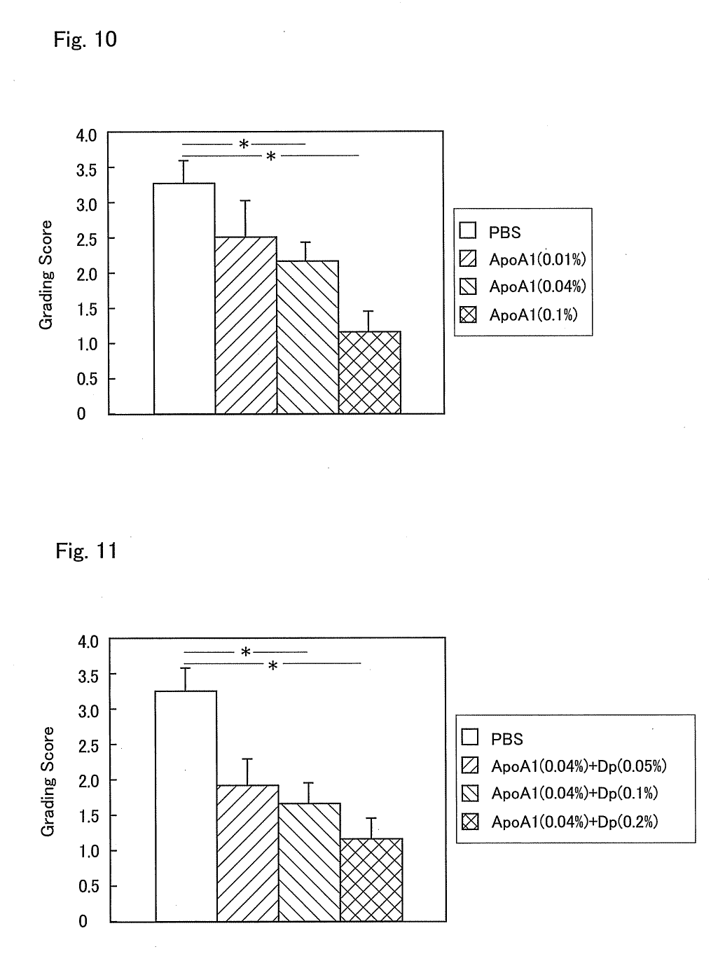

In FIG. 10, there is shown a graph of corneal staining scores of DE mice treated with PBS or with eye drops including 0.01 w/v %, 0.04 w/v %, or 0.1 w/v % of ApoA1. The graph is shown to observe effects of various concentrations of ApoA1. Each column of the data represents the mean grading score of each group (mean±SD; n=12 eyes for the PBS treated group, n=6 eyes for the 0.04 w/v % of ApoA1 treated group, and n=3 eyes for the remaining drug-treated groups; and p value (*)<0.01).

According to FIG. 10, 0.1 w/v % is the most effective concentration of ApoA1.

(2-4) Effect of Various Concentrations of D-pantethine

FIG. 11 is a graph showing corneal staining scores of DE mice treated with PBS or with several different eye drops including a fixed concentration of ApoA1 and respective various concentrations of D-pantethine, so as to observe the effects of various concentrations of D-pantethine. In the eye drops, the concentration of ApoA1 was fixed to 0.04 w/v %, while the concentrations of D-pantethine were 0.05 w/v %, 0.1 w/v %, and 0.2 w/v %, respectively. Each column of the data represents the mean grading score of each group (mean±SD; n=12 eyes for the PBS treated group, n=6 eyes for the ApoA1 (0.04 w/v %) and D-pantethine (0.05 w/v %) treated group, and n=3 eyes each for the remaining drug-treated groups; and p (*) value <0.01).

According to FIG. 11, ApoA1+D-pantethine (0.04 w/v %±0.2 w/v %) is the most effective concentration.

As a conclusion of the above experiments of corneal fluorescein staining, there was indicated that ApoA1 is significantly effective for the treatment of dry eye syndrome in dose-dependent manner. In addition, by using D-pantethine together with ApoA1, the effect of ApoA1 is synergistically enhanced. This action may be seen in a dose-dependent manner.

(3) Histopathologic Study

Mice eyeballs were surgically excised, and fixed with neutral-buffered formalin (10%) and embedded in paraffin. Then, 5 μm thick histological sections were cut and stained with hematoxylin and eosin. These sections of the eyes were examined and photographed with a Provis AX 70 microscope (Olympus Optical Co. Ltd., Japan) equipped with a CAMEDIA digital camera, C-4040 Zoom (Olympus Optical Co. Ltd., Japan). Microphotometric measurement of the thickness was done by using OB-M, 1/100, micro, AX0001 (Olympus Optical Co. Ltd., Japan). The corneal epithelial thickness was measured to an accuracy of 10 μm.

(3-1) Effect of Eye Drops Including ApoA1

In this EXAMPLE, there were used 6 to 8 corneas of each of the following groups of mice: (a) normal mice, (b) DE mice, (c) DE mice treated with 0.04 w/v % of ApoA1, and (d) DE mice treated with PBS.

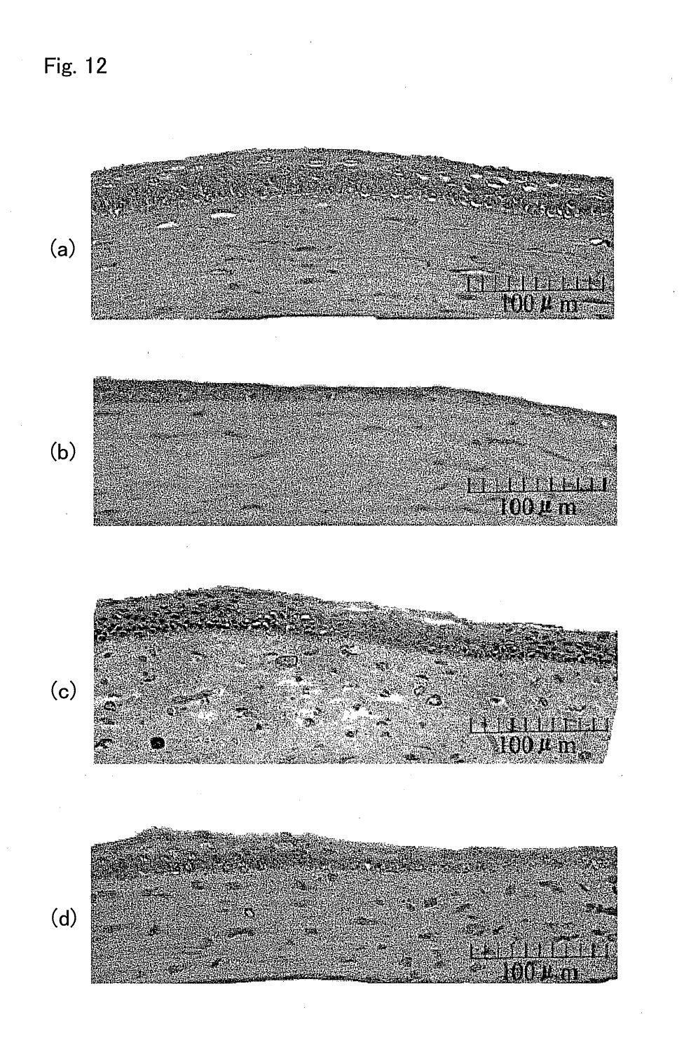

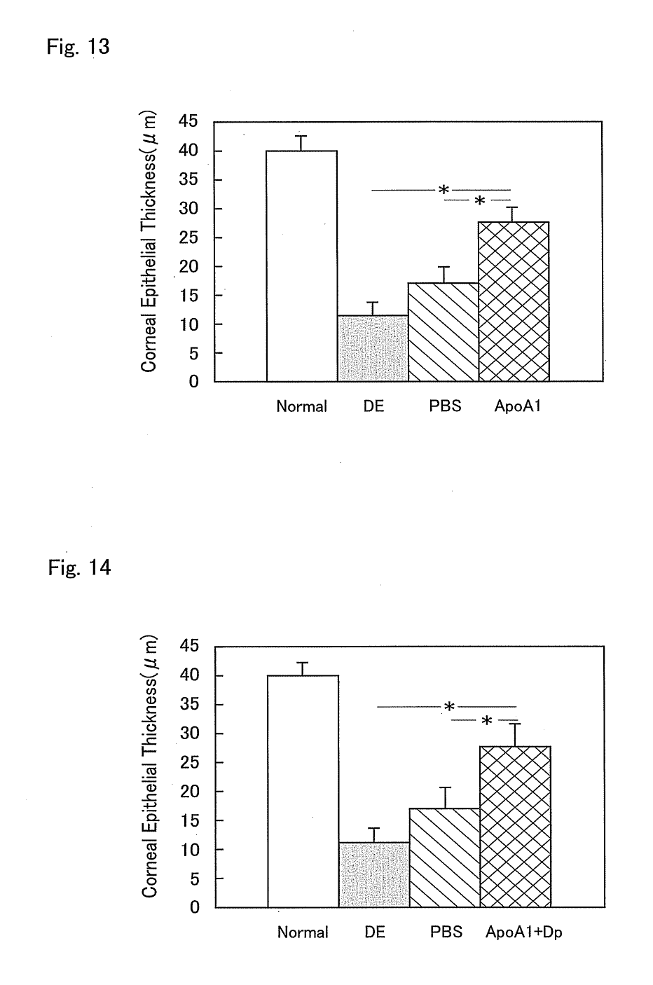

In FIG. 12, there is shown a photograph (magnification of 100) of a representative piece of corneal epithelium of each group. Meanwhile, in FIG. 13, there is shown a graph of average corneal epithelial thickness of the respective groups of mice (mean±SD, n=6 to 8 corneas, and p (*)<0.01).

According to FIGS. 12 and 13, corneal epithelium of DE mice is remarkably thinner than that of the untreated control mice. Thinning of the cell layers accompanied by detachment of the corneal epithelium was significantly greater in the PBS-treated eyes compared with the ApoA1-treated eyes. There is recognized that the barrier function of the lipid layer which constitutes the tear film is enhanced, whereby the corneal epithelium was protected, owing to the treatment with ApoA1.

(3-2) Effect of Eye Drops Including ApoA1 and D-pantethine

In FIG. 14, there is shown a graph of respective average corneal epithelial thicknesses of the groups of normal mice, DE mice, DE mice treated with PBS, and DE mice treated with 0.04 w/v % of ApoA1 and 0.05 w/v % of D-pantethine (mean±SD, n=6 to 8 corneas, and p (*)<0.01).

According to FIG. 14, the group of mice which were treated with 0.04 w/v % of ApoA1 and 0.05 w/v % of D-pantethine exhibited a significant result.

(3-3) Effect of Various Concentrations of ApoA1

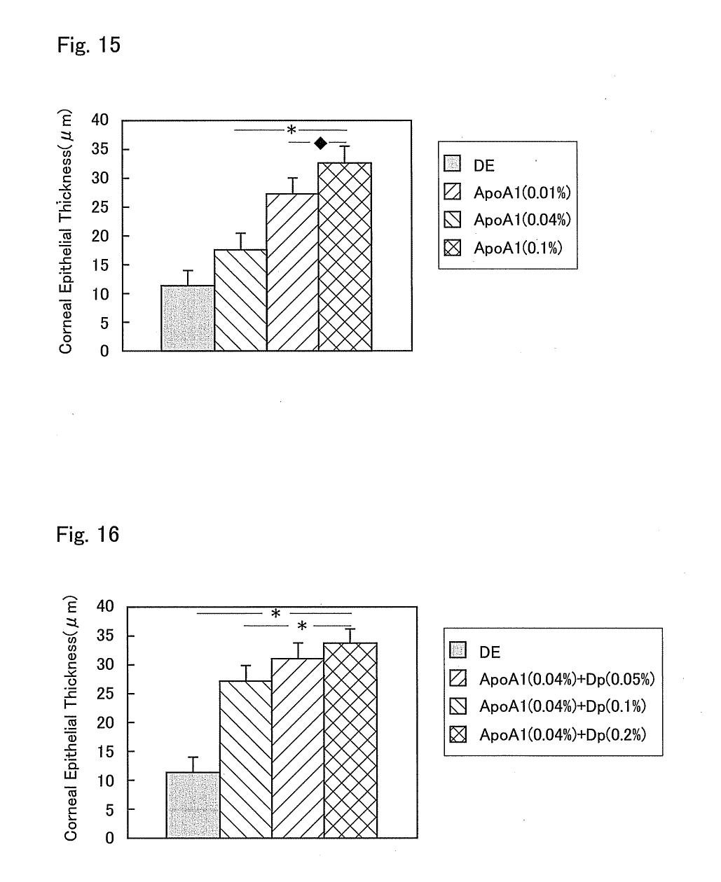

In FIG. 15, there is shown a graph of respective average corneal epithelial thicknesses of the group of DE mice and of the group of DE mice treated with various eye drops, in which the concentrations of ApoA1 were 0.01 w/v %, 0.04 w/v %, and 0.1 w/v %, respectively (mean±SD, n=3 to 8 corneas, and p values are: *<0.01 and ♦<0.05).

According to FIG. 15, 0.1 w/v % is the most effective concentration of ApoA1.

(3-4) Effect of Various Concentrations of D-pantethine

In FIG. 16, there is shown a graph of respective average corneal epithelial thicknesses of the group of DE mice and the group of DE mice treated with several different eye drops including a fixed concentration of ApoA1 and respective various concentrations of D-pantethine, so as to observe the effects of various concentrations of D-pantethine. In the eye drops, the concentration of ApoA1 was fixed to 0.04 w/v %, while the concentrations of D-pantethine were 0.05 w/v %, 0.1 w/v %, and 0.2 w/v %, respectively (mean±SD, n=3 to 8 corneas, and p (*) value <0.01).

According to FIG. 16, the effect of ApoA1 is synergistically enhanced by the addition of D-pantethine, and ApoA1+D-pantethine (0.04 w/v %±0.2 w/v %) is the most effective concentration.

As a conclusion of the above experiments of measurement of the corneal epithelial thicknesses, there is indicated that ApoA1 is extremely effective for the restriction of defections of the corneal epithelium cells (thinning of corneal epithelium) caused by dry eye or keratoconjunctivitis. In addition, by using D-pantethine together with ApoA1, the effect of ApoA1 is synergistically enhanced. This action may be seen in a dose-dependent manner.

Based on the above indicated data of the in vitro and in vivo experiments, there can be concluded that the eye drops including ApoA1 provide excellent influences to pathogenesis of dry eye and keratoconjunctivitis. In addition, according to the above findings, it seems that the ophthalmic composition including ApoA1 can be clinically applied to ease various corneal/conjunctival disorders such as dry eye, keratoconjunctivitis, allergic conjunctivitis, and microbial keratitis.

What is claimed is:

1. An ophthalmic composition comprising an apolipoprotein A-1 as an active ingredient.

2. The ophthalmic composition according to claim 1, further comprising at least one vitamin B5 compound.

3. The ophthalmic composition according to claim 2, wherein the at least one vitamin B5 compound is D-pantethine.

4. The ophthalmic composition according to claim 1, wherein the ophthalmic composition is used for a treatment of corneal disorder.

5. The ophthalmic composition according to claim 4, wherein the corneal disorder is dry eye.

6. The ophthalmic composition according to claim 1, wherein the ophthalmic composition is used as eye drops.

7. The ophthalmic composition according to claim 1, wherein the ophthalmic composition is used as a solution for a contact lens.

8. The ophthalmic composition according to claim 6, wherein the apolipoprotein A-1 is included in the composition in a concentration of 0.001 to 1 w/v %.

9. The ophthalmic composition according to claim 7, wherein the apolipoprotein A-1 is included in the composition in a concentration of 0.001 to 1 w/v %.

10. The ophthalmic composition according to claim 6, wherein at least one vitamin B5 compound is included in the composition in a concentration of 0.005 to 2 w/v %.

11. The ophthalmic composition according to claim 7, wherein at least one vitamin B5 compound is included in the composition in a concentration of 0.005 to 2 w/v %.
